(19) United States
(12) Patent Application Publication
(10) Pub. No.: US 2005/0063907 A1
(43) Pub. Date: Mar. 24, 2005

(54) Radioopaque sustained release pharmaceutical system

(76) Inventors: Malcolm Brandon, Bulleen Victoria (AU); Serge R. Martinod, Groton, CT (US)

Correspondence Address: MORGAN LEWIS & BOCKIUS LLP, 1111 PENNSYLVANIA AVENUE NW, WASHINGTON, DC, 20004, US

(21) Appl. No.: 10/495,642

(22) PCT Filed: Dec. 9, 2002
(86) PCT No.: PCT/AU02/01661

(30) Foreign Application Priority Data
Dec. 14, 2001 (AU) PR 9515

(51) Int. Cl. A61K 49/04

(52) U.S. Cl. 424/9.4

(57) ABSTRACT

An at least partially radio-opaque sustained release delivery apparatus including a sustained release support material; a pharmaceutically active composition carried in or on the sustained release support material; and a material which renders the delivery apparatus at least partially radio-opaque; the pharmaceutically active composition including at least one pharmaceutically active component; and optionally carrier therefor; the pharmaceutically active component being present in amounts of from approximately 30% to 75% by weight, based on the total weight of the sustained release delivery apparatus; the radio-opaque material being carried in the support material, and/or in the pharmaceutically active composition.

The present invention relates to sustained release pharmaceutical compositions, and in particular a method for the preparation thereof. More specifically, the present invention relates to a sustained release pharmaceutical composition, which provides a significant increase in pharmaceutical payload, and is modified to be at least partially radio-opaque.

A number of drug delivery systems are known in the prior art.

For example, a controlled drug-release preparation using as a carrier a hydrophobic polymer material, which is non-degradable after administration into the living body. There are two methods of controlling release of a drug from such preparation; one, using an additive such as an albumin (Japanese patent publication (Tokkohei) No. 61959/1995), and another, by forming an outer layer consisting of hydrophobic polymer alone (Japanese patent publication (Tokkaihei) No. 187994/1995).

However, where a disease indication requires the achievement of a high threshold blood plasma level and/or requires the delivery of multiple pharmaceuticals and/or requires sustained release to be continued over an extended period, the drug delivery systems known in the prior art generally exhibit insufficient drug carrying capacity.

Sustained release delivery apparatuses which can be implanted into a living body offer a major advantage over injections since, in the event of side effects caused by pharmaceutical actives, the implant can be removed. However, it is sometimes difficult to locate the implants quickly by palpation.

It has been found that implants can be made radio-opaque by addition of materials, including barium sulfate, so that, e.g. X-rays may be used to locate the implants in soft tissue.

Some silicone tubing used in medical devices known in the prior art are made radio-opaque by adding barium sulfate as a stripe, typically 20% of the outer diameter. The present invention differs from the prior art tubing because the outer covering of barium sulfate covers 100% of the outer diameter in a very thin layer and the implant is used to deliver drugs.

In addition, techniques known in the prior art for producing sustained release implants utilise a silicone based technology based on an extrusion or molding system.

Difficulties have been encountered in attempting to scale up such techniques to commercial volumes. Difficulties have also been encountered in applying such extrusion techniques to pharmaceutical actives such as Ceftiofur and Recombinant Porcine Somatotropin (rPST). For example, such activities interfere with silicone chemistry due to their chemical composition or exhibit temperature sensitivity.

It is accordingly, an object of the present invention to overcome or at least alleviate one or more of the difficulties and deficiencies related to the prior art.

Accordingly, in a first aspect, there is provided an at least partially radio-opaque sustained release delivery apparatus including
a sustained release support material;
a pharmaceutically active composition carried in or on the sustained release support material; and
a material which renders the delivery apparatus at least partially radio-opaque;
the pharmaceutically active composition including
   at least one pharmaceutically active component; and optionally
   a carrier therefor;
the pharmaceutically active component being present in amounts of from approximately 30% to 75% by weight, preferably approximately 35% to 65% by weight, more preferably approximately 40% to 50% by weight, based on the total weight of the sustained release delivery apparatus;
the radio-opaque material being carried in the support material, and/or in the pharmaceutically active composition.

The sustained release delivery apparatus may take the form of a covered rod or dispersed matrix structure. The sustained release apparatus may take the form of a mini-implant, pellet or tablet.

In addition, the inclusion of a radio-opaque material permits the implant to be quickly located for removal, monitoring and the like.

The sustained release support material may be formed from a biodegradable or biocompatible material, preferably a biocompatible hydrophobic material. The biocompatible material may be selected from the group consisting of polyesters, polyamino acids, silicones, ethylene-vinyl acetate copolymers and polyvinyl alcohols. Preferably the sustained release support material is a silicone material. A silicone rod is preferred. The silicone material may be a porous silicone or a Biosilicon material, for example as described in International patent application PCT/GB99/01185, the entire disclosure of which is incorporated herein by reference. A mesoporous, microporous or polycrystalline silicon or mixtures thereof may be used.

Biodegradable polymers that may be employed in the present invention may be exemplified by, but not limited to, polyesters such as poly(lactic acid-glycolic acid) copolymers (PLGA), etc. and by hydrophobic polyamino acids such as polyaranin, polyleucine etc., polyanhydride, collagen and the like. The hydrophobic polyamino acids mean polymers prepared from hydrophobic amino acids.

Nonbiodegradable polymers that may be employed in the present invention may be exemplified by, but not limited to, silicones, polytetrafluoroethylenes, polyethylenes, polypropylenes, polyurethanes, polyacrylates, polymethacrylates such as polymethylmethacrylates, etc., ethylene-vinyl acetate copolymers, and others. More preferably, a silicone, for example Silastic™ Medical Grade ETR Elastomer Q7-4750 or Dow Corning® MDX 44210 Medical Grade Elastomer, is employed for the corresponding ease of molding.

In a preferred aspect of the present invention the sustained release support material may be formed from a silicone base polymer. The silicone base polymer may be of any suitable type. A biocompatible silicone base polymer is preferred. A methyl/vinyl silicone polymer is preferred.

A reinforcing filler, e.g. a fumed silica, may be included in the silicone base polymer. A silicone elastomer including fumed silica sold under the trade designations CS10401 or CS10701, and blends thereof, available from IMMIX Technologies LLC, Cri-Sil Division, have been found to be suitable.

The silicone base polymer component may be present in amounts of from approximately 15 to 80% by weight, preferably greater than 25% by weight, based on the total weight of the sustained release apparatus. The silicone base polymer can be either liquid form or “gum stock.” Preference is dictated by the type of process used to form and coat the sustained release apparatus. Blending of multiple forms is a typical procedure for obtaining the desired physical properties.

The sustained release delivery apparatus according to the present invention preferably exhibits loading capacities of pharmaceutical active of 30% to 65% by weight, more preferably 35% to 50% by weight, most preferably approximately 40% to 50% by weight, based on the total weight of the pharmaceutically active composition.

Such increased loading capacity permits the treatment of diseases over an extended period with pharmaceutically active components which have heretofore not been applicable to such diseases as it has not been possible to achieve the required threshold blood plasma levels to be efficacious and to maintain those blood levels over an extended period of time.

Preferably the sustained release delivery apparatus may provide approximately zero order release of pharmaceutical active.

For example, in veterinary applications, the pharmaceutically active component ivermectin is a mixture of not less than 90% ivermectin H₂B₁a and not more than 5% ivermectin H₂B₁b having the respective molecular weights 875.10 and 861.07. Ivermectin is a potent macrocyclic lactone disaccharide antiparasitic agent used to prevent and treat parasite infestations in animals. The compound has activity against both internal and external parasites as well as being effective against arthropods, insects, nematodes, filarioidea, platyhelminths and protozoa.

Other macrocyclic lactones which may be used include moxidectin, eprinomectin, doramectin or mixtures thereof.

Accordingly, in a preferred aspect there is provided an at least partially radio-opaque sustained release delivery apparatus including
a sustained release support material;
an anthelmintic composition carried in or on the support material; and
a material which renders the delivery apparatus at least partially radio-opaque;
the anthelmintic composition including
   an anthelmintic component; and optionally
   a carrier therefor;
the anthelmintic component being present in amounts greater than approximately 30% by weight, preferably approximately 35% to 55% by weight, more preferably approximately 40% to 50% by weight, based on the total weight of the delivery apparatus;
the radio-opaque material being carried in the sustained release support material, and/or in the pharmaceutically active composition.

The anthelmintic component preferably includes a macrocyclic lactone, more preferably ivermectin.

The sustained release support material may be formed from a biodegradable or biocompatible material. The sustained release support material may be formed from a silicone elastomer. The sustained release support material may include a liquid silicone as described above.

The radio-opaque material may include, or be formed of any suitable material, which is itself opaque to X-rays or can render the delivery apparatus opaque. A biocompatible or biodegradable material is preferred. For example the radio-opaque material may include a radio-opaque non-toxic salt or oxide of a heavy metal atom, e.g. barium sulfate, zirconium dioxide, bismuth trioxide, and bismuth subcarbonate. Another material which may be used in the present invention is tungsten. The concentration of the radio-opaque material in the sustained release delivery apparatus may be up to 30% w/w.

In a preferred aspect, both the support material and the radio-opaque material are formed from biodegradable or biocompatible materials.

The anthelmintic carrier, when present, may include standard carrier components as described below.

The sustained release support material may be of any suitable form. The sustained release support material may take the form of a support matrix or rod, preferably a covered rod structure.

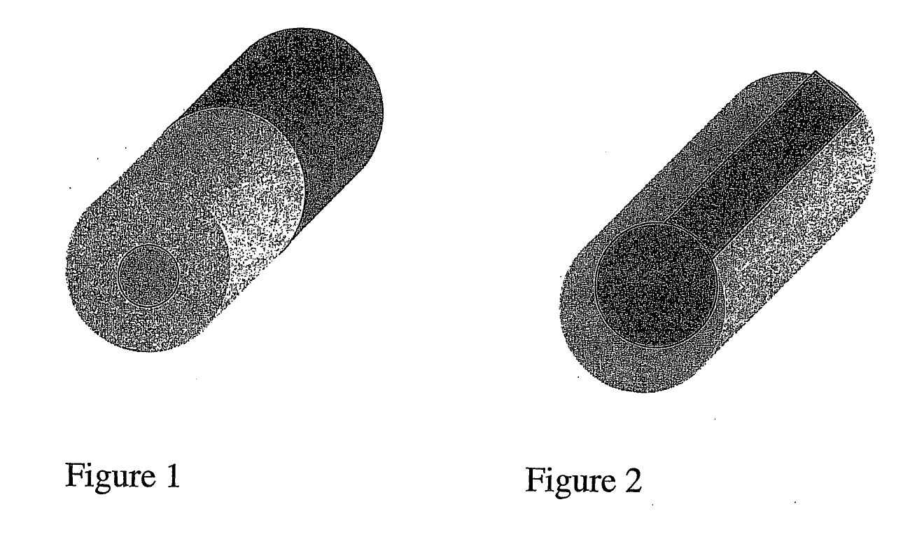

A partially covered rod may be used. Such a structure permits further modification of the release characteristics of the sustained release delivery apparatus according to the present invention. An eccentric or asymmetric rod, optionally partially or fully covered, may be used. Illustrative examples thereof are provided in FIGS. 1 and 2 below.

The sustained release support material may take the form of an open ended cylindrical rod of the type described in U.S. Pat. No. 5,851,547, the entire disclosure of which is incorporated herein by reference.

In a preferred aspect of the present invention the sustained release delivery apparatus may take the form of a biocompatible article suitable for insertion into the body of an animal to be treated.

The biocompatible article may include a medical instrument, apparatus or prosthetic device, or part thereof.

For example, the biocompatible article may include a catheter, or prosthetic appliance, or medical implant, e.g. for reconstructive, dental or cosmetic surgery. Implant materials for replacing or filling bone or like defects are particularly preferred.

It will be understood that by incorporating a pharmaceutically active composition in or on such biocompatible articles, a sustained therapeutic effect may be achieved at the site of insertion.

For example, heparin, an anti-coagulation agent, may be included as the pharmaceutically active component on, or in, e.g a catheter, thus reducing the possibility of blood clots during surgical or other medical procedures.

Similarly, verapamil, an anti-anginal agent, may be included in biocompatible article such as synthetic heart valves, arterial implants or the like as a prophylactic treatment against anginal attacks.

Growth factors, e.g. nerve growth factors, may similarly be included, for example to assist the healing process, e.g. after surgical procedures.

Accordingly, in a further aspect of the present invention, there is provided a process for the preparation of an at least partially radio-opaque sustained release delivery apparatus including
a sustained release support material;
a pharmaceutically active composition including
   at least one pharmaceutically active component; and optionally
   a carrier therefor;
a material which renders the delivery apparatus at least partially radio-opaque;
the pharmaceutically active component being present in amounts of from approximately 30% to 75% by weight, preferably approximately 35% to 65% by weight, based on the total weight of the sustained release delivery apparatus, which process includes
providing
   a silicone base polymer;
   a material which renders the delivery apparatus at least partially radio-opaque;
   a cross-linking agent;
   a pharmaceutically active component; optionally a pharmaceutical carrier
   a catalyst component;
   a curing inhibitor;
mixing the components; and
pre-mixing at least a portion of the silicone base polymer, the pharmaceutical carrier, catalyst, and/or radio-opaque material together to form a first part;
pre-mixing the cross-linking agent, any remaining silicone base polymer, a curing inhibitor and pharmaceutical active and/or the radio-opaque material for a time sufficient to at least partially wet the pharmaceutical active and form a second part; and
feeding the mixtures into a molding apparatus or extruder at a temperature for a time sufficient to permit the components to cure to form the sustained release delivery apparatus.

Temperatures between approximately 15° C. to 350° C. may be used.

It has surprisingly been found that the use of the process according to the present invention permits preparation of a sustained release delivery apparatus with significantly increased payloads.

As the process may be conducted at, or below, 200° C., the method may be applied to the preparation of delivery systems for pharmaceutical actives including sensitive, particularly heat-sensitive, pharmaceutical actives. The duration of the curing step may range from 30 seconds to 180 minutes depending upon the type of process used. For heat-sensitive actives, a curing time of approximately 5 to 30 minutes at a temperature below the degradation temperature, preferably approximately 7.5 to 15 minutes, more preferably approximately 10 to 12 minutes, may be used.

Pharmaceutical actives, e.g. sulfur-containing pharmaceuticals, which heretofore could not be used, e.g. due to fouling of the metal catalyst, may be used in the process according to the present invention.

Such curing conditions are preferably achieved utilising a metal catalyst, more preferably a platinum catalyst, as described below.

The curing inhibitor may be an acetylinic alcohol. The amount of inhibitor used is dependent on the curing temperature selected, the lower the temperature the lower the concentration of inhibitor required. A concentration in the amount of 0 to approximately 2% by weight may be used.

The radio-opaque material may be any non-toxic salt or oxide of a heavy metal atom which is opaque to X-rays, including barium sulfate or zirconium dioxide. The concentration of the radio-opaque material in the sustained release delivery apparatus may be from approximately 0.5 to 30% by weight, preferably approximately 0.5 to 5% by weight, more preferably approximately 1 to 2% by weight, based on the total weight of the sustained release delivery apparatus.

As stated above, the process of preparing the sustained release apparatus is a multi-step process; e.g. pre-mix, mix, form, cure, and coat. This permits the composition to be mixed thoroughly with the sustained release support material before the pharmaceutical active and catalyst are brought into contact.

Accordingly, pharmaceutical actives, e.g. sulfur containing chemicals, which heretofore could not be used, e.g. due to inhibition of silicone curing, may be used in the process according to the present invention.

By utilising a pre-mixing step, potential interference between the pharmaceutical active and catalyst may be reduced or minimized. The pre-mixing process also enables more thorough dispersion of the pharmaceutical actives and carriers without adding to the “work-time” of the final silicone mixture.

In a preferred form, where the pharmaceutically active component does not tend to inhibit the silicone curing process, at least a portion of the active may be included in the first part. This is preferred where a high loading capacity of active is to be achieved.

In the process according to the present invention, the support material may be formed from a biodegradable or biocompatible material. The support material may be formed from a silicone base polymer. The silicone base polymer may be of any suitable type. A biocompatible silicone base polymer is preferred. A methyl/vinyl silicone polymer is preferred.

Injection-molding processes may utilize up to 100% liquid silicone base polymer. Compression-molding or transfer-molding may utilise approximately 0.5 to 20% by weight, preferably approximately 2.5 to 7.5% by weight of a liquid silicone component.

The cross-linking agent utilised in the process according to the present invention may be of any suitable type. A siloxane polymer; e.g. a partially methylated polysiloxane polymer, may be used.

The radio-opaque material may include, or be formed of any suitable material, which is itself opaque to X-rays or can render the delivery apparatus opaque. A biocompatible or biodegradable material is preferred. For example the radio-opaque material may include a radio-opaque non-toxic salt or oxide of a heavy metal atom, e.g. barium sulfate, zirconium dioxide, bismuth trioxide, and bismuth subcarbonate. Another material which may be used in the present invention is tungsten.

The pharmaceutically active composition, as described above, may include
at least one pharmaceutically active component; and optionally
a carrier therefor.

The pharmaceutically active component may include a water-insoluble pharmaceutical, a water-soluble pharmaceutical, a lipophilic pharmaceutical, or mixtures thereof.

The pharmaceutically active component may be exemplified by, but not limited to, one or more selected from the group consisting of:

| | |
|---|---|
| Antiviral pharmaceuticals | Acetonemia preparations |
| Analgesics | Anabolic agents |
| Anti-arthritic | Anaesthetics |
| Antibodies | Anti-acid |
| Anti-infectives | Anti-inflammatories |
| Anti-parasitic | Anti-convulsivants |
| Anti-ulcer | Anti-fungals |
| Blood and blood substitutes | Anti-histamine |
| Cancer therapy and related | Anti-microbials |
| Cardiovascular pharmaceuticals | Anti-protozoals |
| Central nervous system pharma | Behaviour modification drugs |
| Contraceptives | Biologicals |
| Contrast agents | Bronchodilators and expectorants |
| Diabetes therapy | Coccidiostats and coccidiocidals |
| Fertility pharmaceuticals | Diuretics |
| Hormone replacement therapy | Growth promoters |
| Natural products | Hematinics |
| Nutraceuticals and nutritionals | Hemostatics |
| Obesity therapeutics | Hormones and analogs |
| Ophthalmic pharmaceuticals | Immunostimulants |
| Osteoporosis drug | Muscle relaxants |
| Over the Counter (OTC) pharma | Minerals |
| Pain therapeutics | Sedatives and tranquilizers |
| Respiratory pharmaceuticals | Urinary acidifiers |
| Transplantation products | Vitamins |
| Vaccines and adjuvants | |

The pharmaceutically active component may include a water-insoluble pharmaceutical, a water-soluble pharmaceutical, a lipophilic pharmaceutical or mixtures thereof.

The pharmaceutically active component may be a heat-susceptible component such as rPST and/or a sulfur-containing component such as ceftiofur.

The water-soluble pharmaceuticals useful in the sustained release delivery apparatus according to the present invention include such drugs as peptides, proteins, glycoproteins, polysaccharides, and nucleic acids.

The present invention is particularly appropriate for pharmaceuticals that are very active even in extremely small quantities and whose sustained long-term administration is sought. When used in substantially increased quantities, such pharmaceuticals may be applied to disease indications heretofore untreatable over an extended period. The pharmaceuticals may be exemplified by, but not limited to, one or more selected from the group consisting of cytokines (eg. interferons and interleukins), hematopoietic factors (eg. colony-stimulating factors and erythropoietin), hormones (eg. growth hormone, growth hormone releasing factor, calcitonin, leuteinizing hormone, leuteinizing hormone releasing hormone, and insulin), growth factors (eg. somatomedin, nerve growth factor), neurotrophic factors, fibroblast growth factor, and hepatocyte proliferation factor; cell adhesion factors; immunosuppressants; enzymes (eg. asparaginase, superoxide dismutase, tissue plasminogen activating factor, urokinase, and prourokinase), blood coagulating factors (eg. blood coagulating factor VIII), proteins involved in bone metabolism (eg. BMP (bone morphogenetic protein)), and antibodies.

The interferons may include alpha, beta, gamma, or any other interferons or any combination thereof. Likewise, the interleukin may be IL-1, IL-2, IL-3, or any others, and the colony-stimulating factor may be multi-CSF (multipotential CSF), GM-CSF (granulocyte-macrophage CSF), G-CSF (granulocyte CSF), M-CSF (macrophage CSF), or any others.

Vaccines are particularly preferred. The vaccines useful in the sustained release delivery apparatus according to the present invention may be exemplified by, but not limited to, one or more selected from the group consisting of

| | |
|---|---|
| Adenovirus | Anthrax |
| BCG | Chlamydia |
| Cholera | Circovirus |
| Classical swine fever | Coronavirus |
| Diphtheria-Tetanus (DT for children) | Diphtheria-Tetanus (tD for adults) |
| Distemper virus | DTaP |
| DTP | E coli |
| Eimeria (coccidosis) | Feline immunodeficiency virus |
| Feline leukemia virus | Foot and mouth disease |
| Hemophilus | Hepatitis A |
| Hepatitis B | Hepatitis B/Hib |
| Herpes virus | Hib |
| Influenza | Japanese Encephalitis |
| Lyme disease | Measles |
| Measles-Rubella | Meningococcal |
| MMR | Mumps |
| Mycoplasma | Para influenza virus |
| Parvovirus | Pasteurella |
| Pertussis | Pestivirus |
| Plague | Pneumococcal |
| Polio (IPV) | Polio (OPV) |
| Pseudorabies | Rabies |
| Respiratory syncitial virus | Rotavirus |
| Rubella | Salmonella |
| Tetanus | Typhoid |
| Varicella | Yellow Fever |

Pharmaceuticals that can be applied in pharmaceutically active compositions according to the present invention may be further exemplified by low-molecular-weight drugs such as water-soluble anticancer agents, antibiotics, anti-inflammatory drugs, alkylating agents, and immunosuppressants. Examples of these drugs include adriamycin, bleomycins, mitomycins, fluorouracil, peplomycin sulfate, daunorubicin hydrochloride, hydroxyurea, neocarzinostatin, sizofiran, estramustine phosphate sodium, carboplatin, beta-lactams, tetracyclines, aminoglycosides, and phosphomycin.

The pharmaceutically active composition of the present invention may contain two or more drugs depending on the disease and method of application.

Water-insoluble pharmaceutically active components which may be utilised in the sustained release delivery apparatus according to the present invention include lipophilic pharmaceuticals.

A lipophilic pharmaceutical may be any lipophilic substance so long as it is, as a form of a preparation, in a solid state at the body temperature of an animal or a human being to which the preparation is to be administered. Lipophilic as herein used means that the solubility of a substance in water is low, which specifically includes the following natures, as described in Pharmacopoeia of Japan 13th Edition (1996): practically insoluble (the amount of more than or equal to 10000 ml of solvent is required to dissolve 1 g or 1 ml of a solute), very hard to dissolve (the amount of more than or equal to 1000 ml and less than 10000 ml of solvent is required to dissolve 1 g or 1 ml of a solute), or hard to dissolve (the amount of more than or equal to 100 ml and less than 1000 ml of solvent is required to dissolve 1 g or 1 ml of a solute).

Specific examples of the lipophilic pharmaceutical include, but are not limited to, antibiotics such as avermectin, ivermectin, spiramycin, and ceftiofur; antimicrobials (eg. amoxicillin, erythromycin, oxytetracycline, and lincomycin), anti-inflammatory agents (eg. dexamethasone and phenylbutasone), hormones (eg. levothyroxine), adrenocorticosteroids (eg. dexamethasone palmitate, triamcinolone acetonide, and halopredone acetate), non-steroidal anti-inflammatory agents (eg. indometacin and aspirin), therapeutic agents for arterial occlusion (eg. prostaglandin E1), anticancer drugs (eg. actinomycin and daunomycin), therapeutic agents for diabetes (eg. acetohexamide), and therapeutic agents for osteopathy (eg. estradiol).

Depending on a disease or a method for application, multiple lipophilic drugs may be contained. In addition to the lipophilic drug having a direct therapeutic effect, the drug may be a substance with a biological activity, and such a substance as promotes or induces a biological activity, which includes an adjuvant for a vaccine, for example saponin. In such a case, incorporation of a vaccine into a preparation results in a sustained release preparation of a vaccine with an adjuvant.

The pharmaceutically active composition is characterised by including an amount of pharmaceutical active component up to 85% by weight, preferably less than approximately 75% by weight, based on the total weight of the sustained release apparatus.

As stated above, the pharmaceutically active composition according to the present invention may further include a carrier for the pharmaceutically active component.

The pharmaceutical carrier may be selected to permit release of the pharmaceutically active component over an extended period of time from the composition.

The carrier may include a water-soluble substance.

A water-soluble substance is a substance which plays a role of controlling infiltration of water into the inside of the drug dispersion. There is no restriction in terms of the water-soluble substance so long as it is in a solid state (as a form of a preparation) at the body temperature of an animal or human being to which it is to be administered, and a physiologically acceptable, water-soluble substance.

One water-soluble substance, or a combination of two or more water-soluble substances may be used. The water-soluble substance specifically may be selected from one or more of the group consisting of synthetic polymers (eg. polyethylene glycol, polyethylene polypropylene glycol), sugars (eg. sucrose, mannitol, glucose, dextran, sodium chondroitin sulfate), amino acids (eg. glycine and alanine), mineral salts (eg. sodium chloride), organic salts (eg. sodium citrate) and proteins (eg. gelatin and collagen and mixtures thereof). A sugar is preferred.

In addition, when the water-soluble substance is an amphipathic substance, which dissolves in both an organic solvent and water, it has an effect of controlling the release of, for example, a lipophilic drug by altering the solubility thereof. An amphipathic substance includes, but is not limited to, polyethylene glycol or a derivative thereof, polyoxyethylene polyoxypropylene glycol or a derivative thereof, a fatty acid ester, a sodium alkylsulfate of sugars, and more specifically, polyethylene glycol, polyoxy stearate 40, polyoxyethylene[196]polyoxypropylene-[67]glycol, polyoxyethylene[105]polyoxypropylene[5]glycol, polyoxyethylene-[160]polyoxypropylene[30]glycol, sucrose esters of fatty acids, sodium lauryl sulfate, sodium oleate, and sodium desoxycholic acid (sodium deoxycholic acid (DCA)).

Polyoxyethylene polyoxypropylene glycol (also called poloxymers as a generic term), sucrose, or a mixture of sucrose and sodium deoxycholic acid (DCA) are preferred.

In addition, the water-soluble substance may include a substance which is water-soluble and has any activity in vivo, such as low molecular weight drugs, peptides, proteins, glycoproteins, polysaccharides, or antigenic substances used as vaccines, i.e. water-soluble drugs.

The pharmaceutical carrier may constitute from approximately 0% to 30% by weight, preferably approximately 15% to 25% by weight based on the total weight of the sustained release delivery apparatus.

The sustained release delivery apparatus may include additional carrier or excipients, fillers, plasticisers, binding agents, pigments and stabilising agents.

Suitable fillers may be selected from the group consisting of talc, titanium dioxide, starch, kaolin, cellulose (microcrystalline or powdered) and mixtures thereof.

Where the sustained release delivery apparatus takes the form of a biocompatible article, e.g. an implant, calcium fillers, e.g. calcium phosphate, are particularly preferred.

Suitable binding agents include polyvinyl pyrrolidine, hydroxypropyl cellulose and hydroxypropyl methyl cellulose and mixtures thereof.

The catalyst may be of any suitable type. A metal catalyst or peroxide is preferred. A platinum- or rhodium-containing catalyst may be used. A platinum-containing catalyst is preferred for medical applications. If a platinum catalyst is used, it may or may not be attached to an organic ligand. The preferred catalyst is dependent upon the choice of inhibitor, concentration of inhibitor, concentration of cross-linker, and the desired curing profile.

The sustained release delivery apparatus of the present invention may have a rod-like shape, for example it is selected from circular cylinders, prisms, and elliptical cylinders. Alternatively, the sustained release apparatus may take the form of a mini-implant, pellet or tablet. When the device will be administered using an injector-type instrument, a circular cylindrical device is preferred since the injector body and the injection needle typically have a circular cylindrical shape, though other shaped objects may be used. For example, dog microchips may be administered using an injector type instrument.

The size of the pharmaceutical formulation of the present invention may, in the case of subcutaneous administration, be relatively small. For example using an injector-type instrument, the configuration may be circular cylindrical, and the cross-sectional diameter in this embodiment is preferably approximately 0.5 to 4.0 mm, more preferably 0.5 to 1.7 mm, and the axial length is preferably approximately 1 to 40 mm, more preferably 10 to 30 mm.

The thickness of the outer layer should be selected as a function of the material properties and the desired release rate. The outer layer thickness is preferably 0.02 mm to 2 mm, more preferably 0.10 mm to 1 mm, and even more preferably 0.15 mm to 0.2 mm.

The ratio of the axial length of the pharmaceutical formulation to the cross-sectional diameter of the inner layer may, in any case, be one or more and is more preferably two or more and most preferably five or more.

Where a double-layer structure is used, the pharmaceutical-containing inner layer and the drug-impermeable outer layer may be fabricated separately or simultaneously. Silicone is known for swelling with water and being gas-permeable.

A pharmaceutical formulation with an open end at one terminal may be fabricated by dipping one terminal of the pharmaceutical formulation into a solution which dissolves the outer-layer material and drying it, or by covering one terminal end of the pharmaceutical formulation with a cap made from the outer-layer material. In addition, the fabrication may comprise insertion of the inner layer into an outer-layer casing with a closed-end at one terminal, which are separately produced, and also formation of the inner layer in said casing.

In a further aspect of the present invention there is provided a method for the therapeutic or prophylactic treatment of a disease condition in an animal (including a human) requiring such treatment, which method includes administering to the animal an at least partially radio-opaque sustained release delivery apparatus including
a sustained release support material;
a pharmaceutically active composition carried in or on the sustained release support material; and
a material which renders the delivery apparatus at least partially radio-opaque;
the pharmaceutically active composition including
   at least one pharmaceutically active component; and optionally
   a carrier therefor;
the pharmaceutically active component being present in amounts of from approximately 30% to 75% by weight, preferably approximately 35% to 65% by weight, based on the total weight of the sustained release delivery apparatus
the radio-opaque material being carried in the sustained release support material, and/or in the pharmaceutically active composition.

As stated above, it has been found that the pharmaceutical payload may be increased by the sustained release delivery apparatus according to the present invention when compared to the prior art. Diseases which were heretofore untreatable may now be treated over an extended period of time utilising the apparatus of the present invention. In addition, the inclusion of a radio-opaque material permits the implant to be quickly located.

For example, in animals suffering from parasitic infections such as fleas, the animals may be treated utilising the sustained release delivery apparatus including an anti-parasitic drug such as a macrocyclic lactone, e.g. ivermectin, moxidectin, eprinomectin, doramectin or mixtures thereof. Heretofore, it was not possible to achieve a required blood concentration threshold to permit treatment of such a parasitic disease utilising a sustained release approach, as the required blood concentration threshold could not be achieved utilising such a mechanism.

In a further preferred form, the method according to this aspect of the present invention permits the treatment, over an extended period, of diseases and related indications heretofore not treatable due to the sensitivity of the pharmaceutical active.

In this form, the sustained release delivery apparatus may take the form of a biocompatible article as described above, e.g. medical apparatus or implant, as sustained release support material.

In an alternative embodiment a growth hormone, e.g. recombinant porcine somatotropin rPST may be administered to an animal. The required blood concentration may be maintained for an extended period.

The method of administration may include subcutaneous or intramuscular injection, intranasal insertion or indwelling intrarectal insertion or indwelling, for example as a suppository or utilising oral administration.

The animals to be treated may be selected from mice, rats, sheep, cattle, goats, horses, camels, pigs, dogs, cats, ferrets, rabbits, marsupials, buffalos, yacks, birds, humans, chickens, geese, turkeys, rodents, fish, reptiles and the like.

The method according to the present invention is particularly applicable to larger animals, e.g. cattle, sheep, pigs, dogs and humans where high dosage levels are required to achieve the prerequisite threshold pharmaceutical active blood levels for successful treatment of selected disease indications.

The present invention will now be more fully described with reference to the accompanying figures and examples. It should be understood, however, that the description following is illustrative only and should not be taken in any way as a restriction on the generality of the invention described above.

In the figures:

FIG. 1 is a diagrammatic representation of an asymmetric covered rod design of a sustained release delivery apparatus according to the present invention.

In the figure, the lighter colour illustrates a 100% silicone covering and the darker colour in the silicone carrier carrying the pharmaceutical active.

FIG. 2 is a diagrammatic representation of an eccentric covered rod design of a sustained release delivery apparatus according to the present invention.

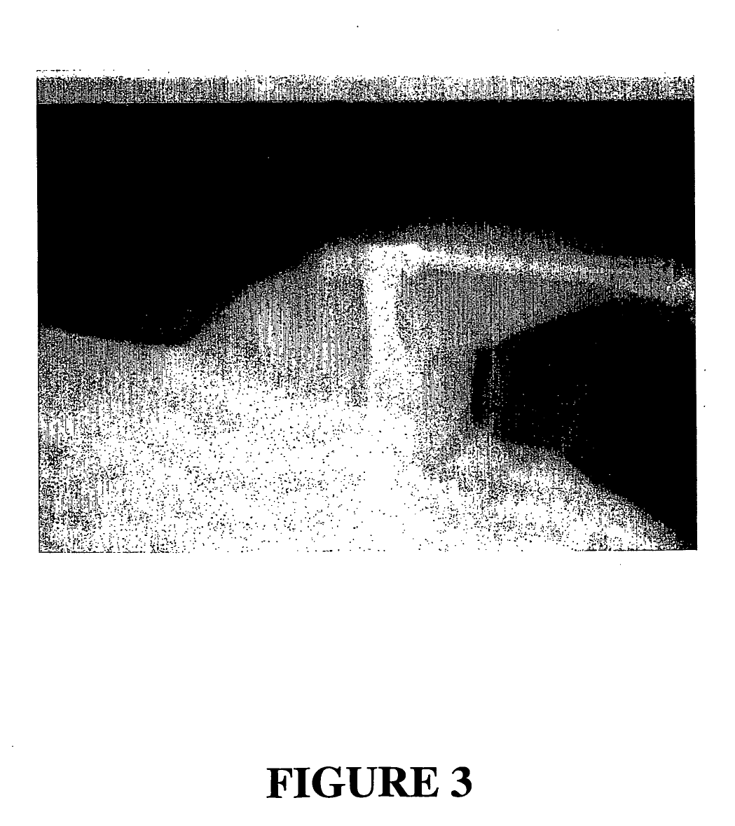

FIG. 3 is a radiograph of a rat showing a sustained release delivery apparatus embedded in the soft tissue.

### EXAMPLE 1

A sample of radio-opaque, covered rod can be prepared using the following procedure:

Prepare a 40% w/w barium sulfate master batch (MB) by mixing the following ingredients on a two-roll mill:
1. 181 g of barium sulfate powder
2. 74 g of Hydride MB (˜33% w/w hydride)
3. 199 g of 40-durometer silicone base polymer (e.g.—CS10401)

Blend the barium sulfate MB in a 50:50 ratio with a “B”-side two-part silicone, containing a similar concentration of cross-linking agent, using a two-roll mill. Preferably of higher durometer than the barium sulfate MB. This will create a “B”-side material that is 20% w/w barium sulfate. This material is then mixed with the “A”-side, containing catalyst and inhibitor, of a two-part silicone. This material is extruded as the outer layer of a co-extruded, covered rod.

The inner material of the co-extruded, covered rod may contain a pharmaceutically active composition. For this example, however, we chose 30% w/w sucrose in both the “A” and “B” sides of the inner material. For our example we chose to extrude a profile with an outer diameter of about 1.60 mm. The outer covering of the co-extruded, covered rod was about 0.18 mm thick.

### EXAMPLE 2

A single Sprague Dawley white laboratory rat was euthanased by an overdose of halothane anaesthetic. The rat was then implanted with a single implant containing a small amount of barium sulfate measuring 1.2 mm in length. The device was implanted subcutaneously on the right side of the rat, near the forelimb. The rat was then positioned in ventral recumbency on a x-ray plate and a radiograph was taken in accordance with standard technique. Two views were taken in this position, and a lateral x-ray was also taken. The x-ray film was developed using an automated processor. The implant was clearly visible on all radiographs (FIG. 3).

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

1-80. (cancelled)

81. An at least partially radio-opaque sustained release delivery apparatus suitable for implant into a living body, said delivery apparatus including
a sustained release support material;
a pharmaceutically active composition carried in or on the sustained release support material; and
a material which renders the delivery apparatus at least partially radio-opaque;
the pharmaceutically active composition including
at least one pharmaceutically active component; and optionally
a carrier therefor;
the pharmaceutically active component being present in amounts of from approximately 30% to 75% by weight, based on the total weight of the sustained release delivery apparatus;
the radio-opaque material being carried in the support material, and/or in the pharmaceutically active composition.

82. A sustained release apparatus according to claim 81, wherein the support material is formed of a silicone base polymer including either a methyl-vinyl polysiloxane polymer or a silicone elastomer including a fumed silica as reinforcing filler, and wherein the silicone base polymer is present in amounts of from approximately 15% to 70% by weight based on the total weight of the apparatus.

83. A sustained release apparatus according to claim 81, wherein the radio-opaque material is selected from one or more heavy metals or non-toxic oxides or salts thereof.

84. A sustained release apparatus according to claim 83, wherein the radio-opaque material is selected from one or more of tungsten, barium sulfate, zirconium dioxide, bismuth trioxide and bismuth subcarbonate.

85. A sustained release apparatus according to claim 81, wherein the radio-opaque material is formed from a biodegradable or biocompatible material.

86. A sustained release apparatus according to claim 85, wherein the support material and radio-opaque material are formed from biodegradable or biocompatible materials.

87. A sustained release apparatus according to claim 81, wherein the pharmaceutically active composition includes a pharmaceutically active component selected from one or more of the group consisting of acetonemia preparations, anabolic agents, anaesthetics, analgesics, anti-acid agents, anti-arthritic agents, antibodies, anti-convulsivants, anti-fungals, anti-histamines, anti-infectives, anti-inflammatories, anti-microbials, anti-parasitic agents, anti-protozoals, anti-ulcer agents, antiviral pharmaceuticals, behaviour modification drugs, biologicals, blood and blood substitutes, bronchodilators and expectorants, cancer therapy and related pharmaceuticals, cardiovascular pharmaceuticals, central nervous system pharmaceuticals, coccidiostats and coccidiocidals, contraceptives, contrast agents, diabetes therapies, diuretics, fertility pharmaceuticals, growth hormones, growth promoters, hematinics, hemostatics, hormone replacement therapies, hormones and analogs, immunostimulants, minerals, muscle relaxants, natural products, nutraceuticals and nutritionals, obesity therapeutics, ophthalmic pharmaceuticals, osteoporosis drugs, pain therapeutics, peptides and polypeptides, respiratory pharmaceuticals, sedatives and tranquilizers, transplantation products, urinary acidifiers, vaccines and adjuvants and vitamins.

88. A sustained release apparatus according to claim 81, wherein the pharmaceutically active component includes an anti-parasitic agent which includes ivermectin.

89. An at least partially radio-opaque sustained release delivery apparatus suitable for implant into a living body, said delivery apparatus including
a sustained release support material;
an anthelmintic composition carried in or on the support material; and
a material which renders the delivery apparatus at least partially radio-opaque;
the anthelmintic composition including
an anthelmintic component; and optionally
a carrier therefor;
the anthelmintic component being present in amounts greater than approximately 30% by weight, based on the total weight of the delivery apparatus;
the radio-opaque material being carried in the support material, and/or in the pharmaceutically active composition.

90. A sustained release apparatus according to claim 89, wherein the anthelmintic component includes ivermectin.

91. A sustained release apparatus according to claim 90, wherein the radio-opaque material is selected from one or more of tungsten, barium sulfate, zirconium dioxide, bismuth trioxide and bismuth subcarbonate.

92. A process for the preparation of an at least partially radio-opaque sustained release delivery apparatus suitable for implant into a living body, said delivery apparatus including
a sustained release support material;
a pharmaceutically active composition; and
a material which renders the delivery apparatus at least partially radio-opaque;
the pharmaceutically active composition including
at least one pharmaceutically active component; and optionally
a carrier therefor;
the pharmaceutically active component being present in amounts of from approximately 30% to 75% by weight, based on the total weight of the sustained release delivery apparatus, which process includes
providing
a silicone base polymer;
a material which renders the delivery apparatus at least partially radio-opaque;
a cross-linking agent;
a pharmaceutically active component; optionally a pharmaceutical carrier
a catalyst component;
a curing inhibitor;
mixing the components; and
pre-mixing at least a portion of the silicone base polymer, the pharmaceutical carrier, catalyst, and/or radio-opaque material together to form a first part;
pre-mixing the cross-linking agent, any remaining silicone base polymer, a curing inhibitor and pharmaceutical active and/or the radio-opaque material for a time sufficient to at least partially wet the pharmaceutical active and form a second part; and
feeding the mixtures into a molding apparatus or extruder at a temperature for a time sufficient to permit the components to cure to form the sustained release delivery apparatus.

93. A process according to claim 92, wherein the silicone base polymer includes a methyl-vinyl siloxane polymer, and a reinforcing filler wherein the reinforcing filler is a fumed silica present in amounts of from approximately 5 to 15% by weight, based on the total weight of the reaction mixture.

94. A process according to claim 92, wherein the pharmaceutically active component includes an anti-parasitic agent which includes ivermectin.

95. A process according to claim 92, wherein the radio-opaque material is selected from one or more heavy metals or non-toxic oxide or salt thereof.

96. A process according to claim 95, wherein the radio-opaque material is selected from one or more of tungsten, barium sulfate, zirconium dioxide, bismuth trioxide and bismuth subcarbonate.

97. A process according to claim 92, wherein the process utilises a co-extrusion apparatus such that the coating layer is deposited concentrically around the apparatus.

98. A biocompatible article including a sustained release apparatus including an at least partially radio-opaque sustained release delivery apparatus suitable for implant into a living body, said delivery apparatus including
a sustained release support material;
a pharmaceutically active composition carried in or on the sustained release support material; and
a material which renders the delivery apparatus at least partially radio-opaque;
the pharmaceutically active composition including
at least one pharmaceutically active component; and optionally
a carrier therefor;
the pharmaceutically active component being present in amounts of from approximately 30% to 75% by weight, based on the total weight of the sustained release delivery apparatus;
the radio-opaque material being carried in the support material, and/or in the pharmaceutically active composition.

99. A biocompatible article according to claim 98, wherein the pharmaceutically active component is present in amounts from approximately 40% to 50% by weight, based on the total weight of the sustained release apparatus.

100. A biocompatible article according to claim 98 wherein the biocompatible article is a medical instrument, apparatus or prosthetic device or part thereof.

101. A biocompatible article according to claim 98, wherein the biocompatible article is a catheter, prosthetic appliance or medical implant for reconstructive dental or cosmetic surgery.

102. A biocompatible article according to claim 101, wherein the biocompatible article is a medical implant material for replacing or filling bone or like defects.

103. A biocompatible article according to claim 98, wherein the pharmaceutically active component includes an anti-coagulation agent.

104. A biocompatible article according to claim 103, wherein the biocompatible article is a catheter.

105. A biocompatible article according to claim 104, wherein the pharmaceutically active component includes an anti-anginal agent.

106. A biocompatible article according to claim 105, wherein the biocompatible article is a synthetic heart valve, arterial implant or part thereof.

107. A biocompatible article according to claim 98, wherein the pharmaceutically active component includes one or more selected from the group consisting of cytokines, hematopoietic factors, hormones, growth factors, neurotrophic factors, fibroblast growth factor, and hepatocyte proliferation factor; cell adhesion factors; immunosuppressants; enzymes, blood coagulating factors, proteins involved in bone metabolism, vaccines and antibodies.

108. A biocompatible article according to claim 107, wherein the pharmaceutically active component includes a nerve growth factor.

109. A biocompatible article according to claim 98 wherein the radio-opaque material is selected from one or more of tungsten, barium sulfate, zirconium dioxide, bismuth trioxide and bismuth subcarbonate.

110. A method for the therapeutic or prophylactic treatment of a disease condition in an animal (including a human) requiring such treatment, which method includes implanting into the animal an at least partially radio-opaque sustained release delivery apparatus including
a pharmaceutically active composition carried in or on the sustained release support material; and
a material which renders the delivery apparatus at least partially radio-opaque;
the pharmaceutically active composition including
at least one pharmaceutically active component; and optionally
a carrier therefor;
the pharmaceutically active component being present in amounts of from approximately 30% to 75% by weight, based on the total weight of the sustained release delivery apparatus
the radio-opaque material being carried in the support material, and/or in the pharmaceutically active composition.

111. A method according to claim 110, wherein the pharmaceutically active component is present in amounts ranging from 40% to 50% by weight, based on the total weight of the apparatus.

112. A method according to claim 110 wherein the pharmaceutically active component includes an anti-parasitic agent which includes ivermectin.

113. A method according to claim 110, wherein the sustained release apparatus forms part of a biocompatible article.

114. A method according to claim 110, wherein the radio-opaque material is selected from one or more of tungsten, barium sulfate, zirconium dioxide, bismuth trioxide and bismuth subcarbonate.
